(19) United States
(12) Patent Application Publication
(10) Pub. No.: US 2021/0290802 A1
(43) Pub. Date: Sep. 23, 2021

(54) CONTAINER-PROCESSING MACHINE AND METHOD FOR CLEANING A CONTAINER-PROCESSING MACHINE

(71) Applicant: KRONES AG, Neutraubling (DE)

(72) Inventors: Merret SACHT, Langballig (DE); Matthias DAWARTZ, Handewitt (DE)

(21) Appl. No.: 17/254,804

(22) PCT Filed: Feb. 20, 2019
(86) PCT No.: PCT/EP2019/054158

§ 371 Date: Dec. 21, 2020

(30) Foreign Application Priority Data
Jun. 26, 2018 (DE) 10 2018 210 369.0

(51) Int. Cl. A61L 2/18; B08B 3/08; B08B 3/02; B08B 9/032; C02F 1/72; C02F 1/32; B65B 55/02; A61L 2/10; A61L 2/26

(52) U.S. Cl. CPC A61L 2/186; B08B 3/08; B08B 3/02; B08B 9/0321; C02F 1/722; C02F 1/325; A61L 2101/02; A61L 2/10; A61L 2/26; B08B 2209/032; C02F 2303/04; C02F 2305/023; B65B 2210/06; B65B 55/025

(57) ABSTRACT

Described is a method for cleaning a tunnel-shaped container-processing machine, in particular a tunnel pasteurizer, and a container-processing machine designed accordingly, in particular a tunnel pasteuriser, for beverage bottles or similar containers. Since hydroxyl radicals are produced mechanically in process water for the container-processing machine the tunnel pasteuriser and/or are introduced into same, and organic impurities present in the container-processing machine in the tunnel pasteuriser are also mineralized by the hydroxyl radicals in the process water, microorganisms based thereon can be removed more easily and in addition their nutrient basis removed from them.

The invention relates to a method according to the preamble of claim 1 as well as a container-processing machine according to the preamble of claim 11.

Bottles made of glass or plastic or also metal cans filled with a beverage can be made hygienic for transport and storage up until consumption in tunnel pasteurizers, in a per se known manner. Such tunnel pasteurizers can comprise both a single deck or decks arranged above one another for treatment of the beverage bottles. The process water used for pasteurization is then collected in collecting tanks, prepared and used again for pasteurization. Sieves, sedimentation tanks as well as filters are used to remove mechanical impurities, for example fragments of broken bottles.

While mechanical and inorganic impurities can readily be removed from the process water using conventional techniques, the organic impurities which are particularly relevant in terms of hygiene in the tunnel pasteurizer are problematic, since they need to be combated comparatively aggressively, for example with disinfectant chemicals. On the one hand, this leads to extended interruptions to production. On the other hand, while undesirable microorganisms such as bacteria and fungi are killed through the use of the chemicals, nonetheless these remain in the interior of the tunnel pasteurizer as biomass. Deposits of biomass, so-called biofilms, which are slimy and correspondingly difficult to remove, on walls, floors and installations within the tunnel pasteurizer are particularly unsightly and unhygienic.

Units are now known with which the recycled process water can be disinfected by means of UV radiation in order to reduce the number of chemical cleaning cycles. However, the undesirable residues of dead biomass can neither be prevented nor removed with these units.

A need thus exists for cleaning methods which are improved in comparison with the above, as well as for tunnel pasteurizers or similar tunnel-shaped container-processing machines with corresponding technical features.

This problem is solved with a method for cleaning an in particular tunnel-shaped container-processing machine, in particular a tunnel pasteurizer, according to claim 1. According to this, hydroxyl radicals are produced mechanically in process water of the container-processing machine, in particular of the tunnel pasteurizer, and/or introduced into same, and organic impurities present in the container-processing machine, in particular in the tunnel pasteurizer, are mineralized by the hydroxyl radicals provided in the process water.

Preferably, the method is carried out in a tunnel pasteurizer and/or tunnel recooler and/or tunnel heater in order to clean same.

The process water is in particular water recycled in the container-processing machine/in the tunnel pasteurizer in order to pasteurize the filled beverages and/or to clean the tunnel pasteurizer.

The mineralized impurities are inorganic and can be flushed out of the container-processing machine and in particular the tunnel pasteurizer comparatively simply, since in contrast to organic impurities and growths of microorganisms, they do not tend to form slimy deposits in the interior of the tunnel pasteurizer. The organic compounds are thereby broken down and at least partially converted into a mineral end product which no longer offers the microorganisms a suitable nutrient basis and therefore also suppresses the growth of microorganisms.

The hydroxyl radicals are produced according to the principle of advanced oxidation, also referred to as the “Advanced Oxidation Process (AOP)”. The end products of the advanced oxidation of organic compounds are known to be non-toxic and environmentally friendly.

The following photochemical processes are known for advanced oxidation: photolysis; UV radiation of H₂O₂ and/or o₃; photo-fenton process through combination of UV radiation, Fe³⁺ and H₂O₂.

Furthermore, the following non-photochemical processes are known for advanced oxidation: ozonation; ozonation in combination with H₂O₂; fenton processes through combination of H₂O₂ with Fe²⁺ or Fe³⁺; wet air oxidation; as well as electrochemical oxidation.

Two fundamental steps are always necessary for the technical implementation of advanced oxidation, i.e. firstly the production of hydroxyl radicals or other powerful oxidizing agents, and then reaction of the hydroxyl radicals with organic impurities which are to be removed.

The reaction of the hydroxyl radicals with the organic impurities can take place in several stages and can comprise the following fundamental reaction types: hydroxyl radicals detach hydrogen atoms from the respective reaction partners, and/or hydroxyl radicals transfer valence electrons to the respective reaction partners, and/or hydroxyl radicals are bound to the respective reaction partners.

Ideally, the advanced oxidation leads to mineral end products, but as a precursor stage thereto can already achieve a helpful suppression of microorganisms in the tunnel pasteurizer through intermediate products, for example through fragmentation and/or derivatisation of organic molecules. Such intermediate products can already be easier to remove and/or offer relevant microorganisms a poorer nutrient basis in comparison with the original organic impurities.

Preferably, the organic impurities comprise microorganisms and/or nutrients for the microorganisms. In this case, the hydroxyl radicals can both break down microorganisms in the container-processing machine and in particular in the tunnel pasteurizer and also suppress their growth through the removal of nutrients. In other words, the production of the microorganisms can both be suppressed and their removal also facilitated following at least partial conversion into inorganic compounds.

Preferably, the hydroxyl radicals are produced photochemically in the region of a preparation unit for process water provided on the container-processing machine and in particular on the tunnel pasteurizer. The light required for the photochemical production process can then be limited to a comparatively small region, for example to a pipeline and/or an irradiation chamber for recycled process water.

Preferably, the hydroxyl radicals are produced in that a photoreactive or UV-reactive oxidizing agent is mixed with the in particular recycled process water and irradiated by means of UV light. The UV light then causes both a disinfection of the prepared process water and also the production of hydroxyl radicals, which dissolve in the process water and can thus be distributed within the interior of the tunnel pasteurizer.

Consequently, the hydroxyl radicals can display their cleaning effect throughout the interior of the tunnel pasteurizer which comes into contact with process water. Photoreactive or UV-reactive oxidizing agents, for example H₂O₂, are comparatively simple to handle, since in this case the hydroxyl radicals are only produced in the irradiation region and can display their effect in a targeted manner thereafter.

Preferably, hydroxyl radicals are produced from H₂O₂. H₂O₂ is a disinfectant widely used to disinfect drinking water supply installations and also in bottling plants for beverages. H₂O₂ is comparatively economical and can readily be mixed with water at room temperature and normal ambient pressure. H₂O₂ can therefore be dosed and introduced simply and precisely in different regions of tunnel pasteurizers.

Preferably, the hydroxyl radicals are produced in the irradiation region of a UV disinfection lamp for process water. On the one hand, the UV light can be used both for the disinfection of the process water and also for the production of the hydroxyl radicals, and thus for a double purpose. The damage to or suppression of microorganisms in the process water caused by the hydroxyl radicals also increases the effectiveness of the disinfection, since the depth of penetration of the UV light in the process water increases with decreasing concentration of the microorganisms. Thus, the disinfection efficiency can be increased with the same electrical lamp output, or a particular disinfection effect may also be achieved with a weaker UV lamp.

In addition or alternatively, the hydroxyl radicals can be produced or introduced non-photochemically and in particular in the region of a collecting tank and/or a sedimentation region for process water provided on the container-processing machine and in particular on the tunnel pasteurizer. This is in particular advantageous if a UV disinfection of process water is not provided or not possible. In this case the method can if necessary also be carried out on existing tunnel pasteurizers with comparatively little expenditure in terms of additional equipment.

Preferably, the cleaning effect of the hydroxyl radicals also involves the molecular fragmentation and/or derivatization of organic impurities. The fragmentation and derivatization are to be understood as intermediate steps in the mineralization of organic impurities through advanced oxidation, but also make their own contribution to the decomposition and/or flushing out of microorganisms.

Preferably, the hydroxyl radicals are produced and/or introduced during the ongoing working operation of the tunnel pasteurizer. For example, the introduction of H₂O₂ into the process water and the subsequent UV disinfection and UV reaction into hydroxyl radicals is possible without any problem during ongoing working operation. Production interruptions for separate cleaning cycles in order to remove microorganisms and/or biomasses/biofilms based thereon from the container-processing machine and in particular the tunnel pasteurizer are thus largely unnecessary.

Preferably, the hydroxyl radicals are dissolved in recycled and prepared process water and distributed within the treatment tunnel of the tunnel pasteurizer by means of spray nozzles. The process water containing the hydroxyl radicals is then distributed within the tunnel pasteurizer via the spray nozzles in order to pasteurize the beverage bottles and/or distributed via additional spray nozzles in order to clean the tunnel pasteurizer. This allows all treatment stages of the tunnel pasteurizer to be cleaned efficiently with the hydroxyl radicals.

The object of the invention is also achieved with an in particular tunnel-shaped container-processing machine and in particular a tunnel pasteurizer according to claim 11. The container-processing machine and in particular the tunnel pasteurizer is suitable for beverage bottles or similar containers, for example also for beverage cans, and comprises at least one preparation unit for process water and spray nozzles for discharging the process water into the container-processing machine and in particular into the tunnel pasteurizer.

Preferably, the container-processing machine comprises a tunnel pasteurizer and/or tunnel recooler and/or tunnel heater or consists of at least one of these units.

The container-processing machine is for example part of a filling plant for beverages or other products with comparable hygiene requirements during production.

According to the invention, the preparation unit is designed for producing and/or introducing hydroxyl radicals into the process water. In this way, the hydroxyl radicals can in particular be discharged effectively into the treatment tunnel of the tunnel pasteurizer and break down and at least partially mineralize organic impurities on the wetted surfaces. The preparation unit is therefore also to be understood as a mineralization unit. The process water can be used to pasteurize the beverage bottles and/or to clean the tunnel pasteurizer.

In this way, unsightly and unhygienic biofilms containing living and dead microorganisms can be removed efficiently from the inner walls of the treatment tunnel, from surfaces of the installations contained therein as well as from pipelines for the process water.

Accordingly, the container-processing machine and in particular the tunnel pasteurizer is designed for carrying out the method according to at least one of the embodiments described above.

Preferably, the preparation unit (mineralization unit) is designed for the non-photochemical production of hydroxyl radicals and/or for the introduction of same into a collecting tank for process water formed in the container-processing machine and in particular in the tunnel pasteurizer and/or for the photochemical production of hydroxyl radicals in a preparation circuit for process water provided on the container-processing machine and in particular on the tunnel pasteurizer. In this way, the non-photochemical and photochemical production of hydroxyl radicals can be carried out particularly efficiently, or also combined with one another.

Preferably, the container-processing machine and in particular the tunnel pasteurizer or the preparation unit comprises at least one UV lamp for the irradiation of process water and a dosing device for a photoreactive or UV-reactive oxidizing agent, in particular H₂O₂, in particular formed in a feed region before the UV lamp, for the photochemical production of the hydroxyl radicals. UV radiation is suitable both for the photochemical production of the hydroxyl radicals as well as for the disinfection of the process water. The oxidizing agent can be dosed precisely and distributed uniformly in the process water in the feed region before the UV lamp before it is immediately thereafter converted into hydroxyl radicals by means of UV light.

Preferably, the dosing device is arranged within a flow length of at most 1 meter upstream of the UV lamp. This prevents the photoreactive or UV-reactive oxidizing agent, for example H₂O₂, which already has an oxidizing effect without UV radiation, already reacting with organic impurities, for example a biofilm, on the way to the UV lamp. Such prior reactions with organic impurities and in particular with microorganisms are undesirable, since they do not lead to any mineralization of the organic impurities with the aforementioned advantages and can instead reduce the efficiency of the mineralization.

Consequently, pipelines with a maximum length of 1 meter between the introduction of the photoreactive oxidizing agents and the entrance to the disinfection chamber illuminated by the UV lamp are particularly suitable. Such a flow distance not exceeding 0.5 meters is particularly advantageous.

Preferably, the UV lamp is part of a preparation unit for the UV disinfection and filtering of process water. The UV lamp can then be used both for disinfection and also for the production of hydroxyl radicals. The production of the hydroxyl radicals can in turn improve the effectiveness of the UV lamp through an improved depth of penetration of the UV light into the process water which is to be disinfected.

Preferably, the preparation unit (mineralization unit) comprises a dosing device for the admixture of a mineralization solution containing hydroxyl radicals with the process water arranged in the region of a collecting tank, in particular of a sedimentation region for the process water. Here, the oxidizing agent can be distributed uniformly in the collected process water and can be returned together with this to the preparation unit. The dosing device could also be arranged in a discharge region from the collecting tank or from the sedimentation region into the preparation unit. In this case the mineralization solution is preferably based on an oxidizing agent for the non-photochemical provision of hydroxyl radicals.

Preferred embodiments of the invention are presented in the drawings, in which:

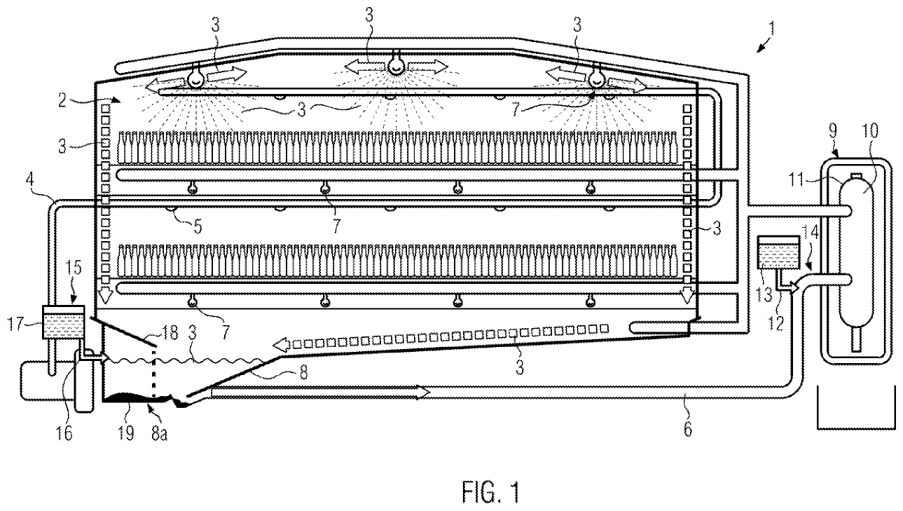

FIG. 1 shows a schematic cross section through a tunnel pasteurizer; and

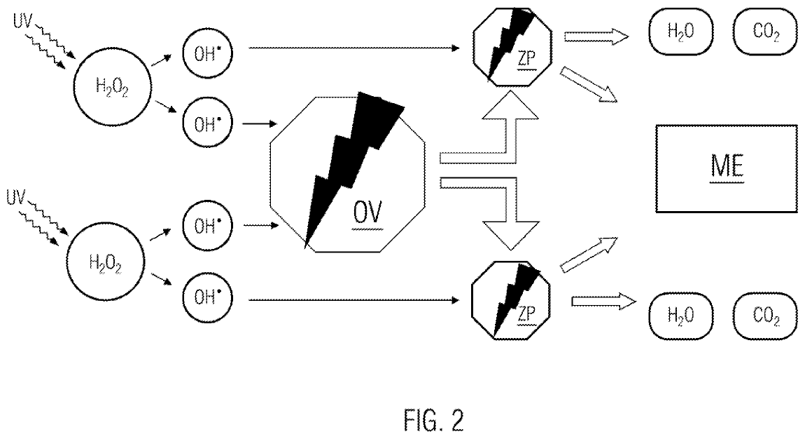

FIG. 2 shows a reaction scheme for the advanced photochemical oxidation of organic impurities.

As can be seen in FIG. 1 in the schematic cross section of a container-processing machine 1, designed by way of example as a tunnel pasteurizer, this comprises a treatment tunnel 2 for spraying beverage bottles or other containers filled with a product with process water 3, which is heated or cooled depending on the treatment zone. This is known, as is the conveying of the beverage bottles on two treatment decks arranged above one another which is illustrated by way of example.

The container-processing machine 1 is tunnel-shaped and can, alternatively or in addition, comprise a tunnel recooler and/or a tunnel heater, or consists of at least one of these units.

The container-processing machine 1 and in particular the tunnel pasteurizer further comprises a treatment circuit 4 with spray nozzles 5 for the treatment of the beverage bottles 2 with the process water 3 as well as a preparation circuit 6 with spray nozzles 7 for cleaning the treatment tunnel 1a with prepared process water 3.

The process water 3 sprayed from the nozzles 5, 7 is collected in a collecting tank 8 and can be drawn off from there both in the direction of the treatment circuit 4, for example for the purpose of heating or cooling, and also in the direction of the preparation circuit 6.

The preparation circuit 6 comprises a first preparation unit 9 for the UV disinfection of the process water 3 and for the photochemical production of hydroxyl radicals in the process water 3 for a subsequent mineralization of organic impurities in the treatment tunnel 2.

The first preparation unit 9 comprises a UV lamp 10 in a disinfection chamber 11 which is flowed through by process water 3 which is to be prepared, and a first dosing device 12 for a photoreactive or UV-reactive oxidizing agent 13, in particular H₂O₂. The first dosing device 12 is preferably arranged in a feed region 14 leading to the disinfection chamber 11 or to the UV lamp 10, in particular within a flow length of not more than 1 meter up to the disinfection chamber 11.

The process water 3 which is to be prepared is irradiated in the disinfection chamber 11 by the UV lamp 10 and as a result disinfected. In addition, the UV lamp 10 causes a reaction of the photoreactive oxidizing agent 13 dosed into the process water 3 by the first dosing device 12 to form hydroxyl radicals. Preferably, the process water 3 is also filtered in a conventional manner in the first preparation unit 9.

The hydroxyl radicals produced photochemically in this way dissolve in the process water 3 and are pumped in this solution via the preparation circuit 6 to the spray nozzles 7. The spray nozzles 7 are distributed in a suitable manner within the treatment tunnel 2 in order to spray the surfaces of inner walls, installations or the like which are to be cleaned with the prepared process water 3.

As a result, the hydroxyl radicals come into contact with all surfaces of the container-processing machine 1 and in particular of the tunnel pasteurizer which are also wetted by the process water 3. Organic impurities present on these surfaces, including microorganisms, react with the hydroxyl radicals introduced in this way and are successively broken down through advanced oxidation and at least partially mineralized. In this way, the formation of slimy and/or solid biofilms adhering to the surfaces of the container-processing machine 1 and in particular of the tunnel pasteurizer is for example counteracted.

Through stepwise fragmentation, derivatisation and mineralization of organic compounds, microorganisms are not only killed off but also broken down into the more easily removed intermediate products and end products of advanced oxidation. Moreover, bacteria, fungi or similar microorganisms are deprived of nutrients. Consequently, a comprehensive reduction and removal of unsightly and unhygienic biofilms in the interior of the container-processing machine 1 and in particular of the tunnel pasteurizer is possible

The first preparation unit 9 thus allows a mechanical and in particular preferably complete mineralization of organic impurities through advanced oxidation in the container-processing machine 1 and in particular in the tunnel pasteurizer.

Such organic impurities can in theory be found throughout the treatment tunnel 2 due to bottles breaking or filled beverages otherwise leaking out. For example, volatile constituents of the beverages can evaporate in the container-processing machine 1 and in particular in the tunnel pasteurizer, not least due to the comparatively high treatment temperatures, and precipitate on the inner walls and/or installations of the treatment tunnel 2. Moreover, good climatic breeding conditions for microorganisms prevail within the treatment tunnel 2 depending on the treatment zone. Thus, biofilms can in principle form in many regions of the treatment tunnel 2. Affected by the organic impurities are for example the collecting tank 8 as well as all pipelines and installations through which process water 3 contaminated with microorganisms flows.

FIG. 1 shows, schematically, the collecting tank 8 for process water 3 formed in the lower region of the treatment tunnel 2 with a sedimentation region 8a in which smaller foreign bodies, suspended particles or otherwise insoluble impurities can settle, as well as a second preparation unit 15 with a second dosing device 16 for admixture of a mineralization solution 17 containing hydroxyl radicals for breaking down organic impurities.

The hydroxyl radicals in the mineralization solution 17 are preferably obtained from a suitable oxidizing agent by means of non-photochemical reaction. However, photoreactive oxidizing agents and/or a photochemical production of the mineralization solution 17 are also conceivable.

The second preparation unit 15 is preferably arranged in the sedimentation region 8a to ensure the uniform intermixing of the process water 3 and the dosed mineralization solution 17. A conventional sieve 18 is for example arranged above the sedimentation region 8a in order to retain glass splinters or other larger foreign bodies from the process water 3 which is to be collected.

The second preparation unit 15 also allows a mechanical and in particular preferably complete mineralization of organic impurities through advanced oxidation in the container-processing machine 1 and in particular in the tunnel pasteurizer.

In the example shown, the first preparation unit 9 is designed for the photochemical production of hydroxyl radicals and their admixture with the process water 3 which is to be prepared, whereas the second preparation unit 15 is designed for the non-photochemical production and/or admixture of hydroxyl radicals with the process water 3. One of the two preparation units 9, 15 can already be sufficient in order to carry out the mineralization of organic impurities.

This is because in both cases the hydroxyl radicals provided in this way cause organic impurities, for example microorganisms and/or nutrients for the microorganisms, to be at least partially converted into mineral end products. These mineral end products can for example settle in the sedimentation region 8a of the collecting tank 8 and are represented in FIG. 1, schematically and by way of example, as a mineral deposit 19. Such mineral deposits 19 can be removed comparatively easily from the container-processing machine 1 and in particular the tunnel pasteurizer with its installations and pipelines, in contrast to biofilms consisting of living and/or dead microorganisms, which due to their slimy consistency can only be removed with great effort and/or only incompletely.

In the context of the present invention, both preparation units 9, 15 can thus also be understood as mineralization units designed to break down microorganisms and resulting dead biomass and convert them, as completely as possible, into intermediate products which can be removed comparatively easily and mineral end products with a low nutrient value for microorganisms.

In comparison with conventional chemical cleaning methods and/or the boiling-out of container-processing machines 1 and in particular tunnel pasteurizers, the quantity of unsightly and hygienically problematic biofilms can be lastingly reduced. In this way, personnel hours and production interruptions for cleaning cycles can be significantly reduced.

Moreover, the preparation units (mineralization units) 9, 15 and the spray nozzles 5, 7 allow the hygienically relevant regions of the container-processing machine 1 and in particular of the tunnel pasteurizer to be cleaned using the process water 3, largely independently of the location of the production and/or introduction of the hydroxyl radicals.

In principle, the photochemical production of hydroxyl radicals would also be possible independently of a UV disinfection/filtering, namely with at least one separate UV lamp (not shown) in the preparation circuit 6 and/or in the treatment tunnel 2. Here too, photoreactive oxidizing agents, for example H₂O₂, dissolved in the process water 3 can in principle be irradiated by means of UV lamps (not shown) and as a result can if necessary also react to form mineralizing hydroxyl radicals directly at the location to be cleaned. In this case the photoreactive oxidizing agent producing the photochemical reaction to form hydroxyl radicals could be dispensed with the process water 3, for example via the treatment circuit 4 and the spray nozzles 5, and irradiated with UV light in a targeted manner at a suitable location.

FIG. 2 shows a fundamental reaction scheme of the advanced oxidation by means of the hydroxyl radicals OH.. By way of example, the advanced oxidation is illustrated starting out from H₂O₂ and UV light UV. According to this, the UV light UV first splits H₂O₂ molecules into two hydroxyl radicals OH..

These then react with an organic impurity OV to form organic intermediate products ZP, in this example through fragmentation of the organic impurity OV. The intermediate products ZP can consist of different organic molecules.

The organic intermediate products ZP can then react, in further reactions with hydroxyl radicals OR, to form one or more mineral end products ME, for example by splitting off H₂O₂ and CO₂.

FIG. 2 is simply intended to convey schematically on which reaction scheme the mineralization of the organic impurities OV is based. The advantageous cleaning effect results from a combination of intermediate steps and the final reaction to form a mineral end product ME. Accordingly, not all organic impurities need to be converted completely into a mineral end product ME. The organic intermediate products ZP, which can for example be produced through the schematically represented fragmentation and/or through derivatisation, are also more easily removed than the organic impurities OV originally present, and moreover deprive microorganisms or similar biomass in the container-processing machine 1 and in particular in the tunnel pasteurizer of nutrients.

The photochemical production of the hydroxyl radicals OR illustrated by way of example is advantageous due to the good handling properties of H₂O₂ in the field of bottling plants or similar installations used within the food industry and the readily controlled use of UV light UV. In particular, the combination with the disinfectant effect of the UV light UV on the process water 3 which is to be prepared is particularly efficient in terms of the operation of the container-processing machine 1 and in particular of the tunnel pasteurizer. On the one hand, the UV lamp 10 can be used simultaneously both to disinfect the process water 3 and also in the production of the hydroxyl radicals OH.. On the other hand, the efficiency of the UV lamp 10 in disinfecting the process water 3 is improved through the decomposition of organic impurities OV.

Both processes, namely the UV disinfection and the photochemical production of the hydroxyl radicals OH., can be carried out during ongoing operation of the container-processing machine 1 and in particular of the tunnel pasteurizer, wherein a comprehensive mineralization and thus reduction in biomass in the container-processing machine 1 and in particular in the tunnel pasteurizer is also achieved through the distribution of the process water 3 in the container-processing machine 1 and in particular in the tunnel pasteurizer.

Both the treatment circuit 4 with its spray nozzles 5 and also the separate preparation circuit 6 with its spray nozzles 7 are suitable for this purpose. Likewise, preparation units (mineralization units) 9, 15 with photochemical and/or non-photochemical production and/or introduction of hydroxyl radicals can be used selectively.

For example, the mineralization of organic impurities OV during ongoing operation can be carried out as follows.

The beverage bottles which are to be pasteurized are fed continuously into the treatment tunnel 2 and conveyed continuously on at least one treatment deck through treatment zones for heating or cooling the beverage bottles. They are thereby sprayed with the process water 3 adjusted to a suitable temperature. The process water 3 runs through the decks and is received by the collecting tank 8 and collected in its sedimentation region 8a. From there, the mechanically clarified process water 3 is drawn off, partially into the preparation circuit 6 and partially into the treatment circuit 4.

In the preparation circuit 6, process water 3 is, at suitable intervals or continuously, pumped to the first preparation unit 9 and irradiated by the UV lamp 10. In the feed region 14 before the UV lamp 10, the photoreactive oxidizing agent 13, in particular H₂O₂, is admixed with the process water 3 in a controlled manner and transported with the process water 3 into the irradiation region of the UV lamp 10. There, the process water 3 is disinfected by means of UV light UV. Hydroxyl radicals OR are thereby formed from the photoreactive oxidizing agent 13. The process water 3 prepared in this way and preferably also mechanically filtered is then pumped to the spray nozzles 7 and distributed within the treatment tunnel 2 by these.

The UV disinfection can in principle thereby be optionally supplemented through the photochemical production of hydroxyl radicals OH. in the process water 3. For example, identical or coordinated preparation and cleaning cycles can be specified for both preparation processes. Likewise, an admixture of the photoreactive oxidizing agents 13 can be activated in the UV disinfection depending on actual requirements or according to a cleaning schedule.

Finally, organic impurities OV present in the irradiation tunnel 2 are exposed to the hydroxyl radicals OH. through wetting with the process water 3 prepared in this way and consequently broken down into organic intermediate products ZP as well as, at least partially, at least one mineral end product ME by means of fragmentation and/or derivatisation.

The intermediate products ZP and the mineral end products ME from the advanced oxidation are in principle soluble in the process water 3 and/or can be carried away mechanically by this and could then settle in the region of the collecting tank 8. This makes possible a continuous or need-oriented cleaning of the container-processing machine 1 and in particular of the tunnel pasteurizer 1 as a whole during ongoing production operation. Growths of microorganisms or biofilms based thereon are removed and/or suppressed.

In addition or alternatively, an in particular non-photochemically-produced mineralization solution 17 containing hydroxyl radicals OH. can be admixed with the process water 3 in a dosed manner, preferably in the region of the collecting tank 8 and in particular its sedimentation region 8a, and in this way distributed as required within pipelines and/or the treatment tunnel 2.

In this way, the mineralization of organic impurities OV can be carried out efficiently, in particular reducing or even preventing growths of microorganisms using at least one of the preparation units (mineralization units) 9, 15. Depending on equipment requirements, these could also be retrofitted in existing tunnel pasteurizers, tunnel recoolers and/or tunnel heaters.

1. Method for cleaning an in particular tunnel-shaped container-processing machine, wherein hydroxyl radicals (OH.) are produced in a process water for the container-processing machine and/or introduced into same, and organic impurities (OV) present in the container-processing machine are mineralized by the hydroxyl radicals provided in the process water.

2. The method according to claim 1, wherein the container-processing machine comprises a tunnel pasteurizer and/or tunnel recooler and/or tunnel heater.

3. The method according to claim 1, wherein the organic impurities comprise microorganisms and/or nutrients for the microorganisms.

4. The method according to claim 2, wherein the hydroxyl radicals (OH.) are produced photochemically in a region of a preparation unit for process water provided on the container-processing machine, in particular on the tunnel pasteurizer.

5. The method according to claim 1, wherein the hydroxyl radicals (OH.) are produced in that a photoreactive oxidizing agent is admixed with in particular recycled process water and irradiated by means of UV light.

6. The method according to one of the preceding claim 1, wherein the hydroxyl radicals (OH.) are produced from H₂O₂.

7. The method according to claim 1, wherein the hydroxyl radicals (OH.) are produced in an irradiation region of a UV lamp for a disinfection of process water.

8. The method according to claim 4, wherein the hydroxyl radicals (OH.) are produced non-photochemically in the region of a collecting tank for process water provided on the container-processing machine, in particular on the tunnel pasteurizer, and/or introduced into same.

9. The method according to claim 2, wherein the hydroxyl radicals (OH.) contained in a recycled and prepared process water are distributed within treatment tunnels of the container-processing machine, in particular of the tunnel pasteurizer, by means of spray nozzles.

10. The method according to claim 2, wherein the hydroxyl radicals (OH.) are produced and/or introduced during ongoing working operation of the container-processing machine of the tunnel pasteurizer.

11. Container-processing machine for beverage bottles or similar filled containers, with a preparation unit for process water and with spray nozzles for discharging the process water into the in particular tunnel-shaped container-processing machine, wherein the preparation unit is designed to produce hydroxyl radicals (OH.) and/or introduce these into the process water.

12. The container-processing machine according to claim 11, wherein the container-processing machine comprises a tunnel pasteurizer and/or tunnel recooler and/or a tunnel heater.

13. The container-processing machine according to claim 12, wherein the preparation unit is designed for a non-photochemical production of hydroxyl radicals (OH.) and/or the introduction of same into a collecting tank formed in the container-processing machine, in particular in the tunnel pasteurizer, and/or for the photochemical production of hydroxyl radicals (OH.) in a preparation circuit for process water provided on the container-processing machine, in particular on the tunnel pasteurizer.

14. The container-processing machine according to one of the claim 11, further having at least one UV lamp for an irradiation of process water and having a dosing device for a photoreactive oxidizing agent formed in a feed region leading to the UV lamp.

15. The container-processing machine according to claim 14, wherein the dosing device is arranged at a distance of no more than 1 meter upstream of the UV lamp.

16. The container-processing machine according to claim 14, wherein the UV lamp is part of a preparation unit for an UV disinfection of process water.

17. The container-processing machine according to claim 14, wherein the preparation unit comprises a dosing device for admixture of a mineralization solution containing hydroxyl radicals (OH.) with the process water arranged in the region of a collecting tank for the process water.
